(19) United States
(12) Patent Application Publication
(10) Pub. No.: US 2004/0116904 A1
(43) Pub. Date: Jun. 17, 2004

(54) Percutaneous plug

(76) Inventor: Manoj Monga, Edina, MN (US)

Correspondence Address: Kenneth K. Vu, 22872 Avenida Empresa, Rancho Santa Margarita, CA, 92688, US

(21) Appl. No.: 10/727,777

(22) Filed: Dec. 3, 2003

Related U.S. Application Data
(60) Provisional application No. 60/431,638, filed on Dec. 5, 2002.

(51) Int. Cl. A61M 27/00; A61M 31/00; A61M 37/00; A61M 29/00

(52) U.S. Cl. 604/544; 604/288.01; 604/93.01; 604/96.01

(57) ABSTRACT

The invention is directed to a percutaneous drainage catheter comprising a tubular member having a drainage lumen extending from a proximal end and a distal end; and a retention member formed around the tubular member and is adapted to move between a low-profile state facilitating insertion of the drainage catheter and a high-profile state facilitating retention of the drainage catheter in a body cavity, wherein the tubular member and the retention member operate to seal and tamponade an access tract in the body cavity. The retention member comprises a soft conforming balloon disposed at the distal end of the tubular member. The drainage lumen provides for drainage of urine, passage of a guidewire, and infusion of liquids. The proximal end of the tubular member is designed to protrude minimally from the body cavity. The drainage catheter further includes an inflation passage to actuate the retention member and to maintain pressure in the retention member for prolonged periods of time. The tubular member may be configured for percutaneous nephrolithotomy or for suprapubic drainage application. The drainage catheter further comprises a drainage portion having at least one drainage port including a Luer-lock connection and a drainage bag. The inflation passage may be connected to a pump or syringe to individually and independently inflate and deflate the retention member. The drainage catheter may further comprise a connector hub at the proximal end including a port and an access lumen plug that operates like a snap-on plug. In another aspect of the invention, the drainage catheter may be used in a veterinary application.

This is a non-provisional application claiming the priority of provisional application Serial No. 60/431,638, filed on Dec. 5, 2002, entitled “Percutaneous Plug,” which is fully incorporated herein by reference.

### BACKGROUND OF THE INVENTION

1. Field of the Invention

This invention generally relates to drainage catheters and, more particularly, to a percutaneous drainage catheter having a snap-on access lumen plug.

2. Discussion of Related Art

Urinary drainage catheters are used to provide an outlet for the urinary bladder when the normal outlet lumen (the urethra) is compromised or obstructed. Such catheters have been used on a more permanent basis, e.g., when a patient is incapable of controlling a capable urinary system due to sedation or lack of mental capacity. A typical urinary catheter is the Foley catheter, which is frequently used for bladder drainage. The Foley catheter is a thick-walled rubber tube with an inflatable balloon near its distal end. The catheter is inserted with the balloon deflated, through the urethra (which extends through the prostate and a bladder neck), and into the bladder cavity. When operably disposed, the balloon is inflated within the bladder cavity to a size that prevents the distal end from passing back through the bladder neck.

Percutaneous Nephrolithotomy (PCNL) is an operation to remove large stones from the kidney. Kidney stones are formed in the urinary tract due to crystallization of chemical compounds in the urine. PCNL is a surgical procedure used to remove certain stones in the kidney or upper ureter. In this procedure, the surgeon makes a tiny incision (1-1.5 cm) in the back of the patient (see, e.g., FIG. 1) and creates a tunnel directly into the kidney (see, e.g., FIG. 2). Using an instrument called a nephroscope, the surgeon can locate and remove the stone. For large stones, an energy probe such as an ultrasonic, electrohydraulic or laser lithotripsy may be needed to break the stone into small pieces. A nephrostomy tube may be placed in the kidney to allow easy drainage of the urine. This drain usually remains for a couple days. A ureteral stent may also be placed in the ureter and extends from the kidney to the bladder to promote drainage from the kidney. The length of time the stent remains in place is variable but is typically removed within 2-6 weeks. The PCNL procedure has resulted in significantly less post-operative pain, a shorter hospital stay and earlier return to work and daily activities.

Suprapubic catherization of the bladder is another technique used to drain the bladder after surgery. In this procedure, a catheter is percutaneously introduced into a patient by a trocar which typically pierces the abdominal wall. A guidewire is inserted through the needle, which is then removed. The catheter tube with a cannula positioned therein is then passed over the guidewire into the cavity. The cannula and guidewire are then withdrawn, leaving the catheter in the desired cavity. FIGS. 3A and 3B illustrate a suprapubic drainage catheter 10 for percutaneous placement in a bladder 12 of a patient for the removal of urine from the bladder 12. The drainage catheter 10 may be placed suprapubically by a mid-line incision through the abdominal wall and into the space behind the pubic bone 14 and into the bladder 12. The drainage catheter 10 will then be sutured into the bladder wall. After implantation, the drainage catheter 10 can be accessed using a percutaneous needle 30 which is connected to a collection or drainage bag 32.

The use of transcutaneous catheters for the drainage of the bladder, the kidney or other body sites, however, is problematic in a number of respects. In particular, the presence of a transcutaneous catheter places the patient at a higher risk of infection because of the long-term percutaneous penetration that is maintained in the patient's skin (e.g., up to or more than 12 weeks). The presence of the external end of the catheter is difficult to maintain in a sterile condition, and its physical presence is a great inconvenience to the patient. In addition, the Foley drainage catheter is uncomfortable to the patient, due particularly to its size and inflexibility. As a result, some patients pull on the end of the catheter causing the catheter to dislodge from the bladder, which may cause traumatic and sometimes damage to the anatomy of the patient. Accordingly, there is a need in the art for an improved percutaneous drainage catheter that is comfortable to the patient and is easy to clean and maintain for prolonged periods of time.

### SUMMARY OF THE INVENTION

The present invention is directed to a percutaneous drainage catheter comprising a tubular member having a drainage or access lumen extending from a proximal end and a distal end; and a retention member formed around the tubular member and is adapted to move between a low-profile state facilitating insertion of the drainage catheter and a high-profile state facilitating retention of the drainage catheter in a body cavity, wherein the tubular member and the retention member operate to seal and tamponade an access or wound tract in the body cavity. The retention member is a soft conforming balloon disposed at the distal end of the tubular member. The tubular member and the retention member in the low-profile state have a diameter of about 8 Fr-10 Fr, and the retention member may be expanded to about 30 Fr in the high-profile state. The drainage lumen or additional lumen may be provided for drainage of urine, passage of a guidewire, and infusion of liquids. The proximal end of the tubular member is designed to protrude minimally from the body cavity. The drainage catheter further includes an inflation passage to actuate the retention member from the low-profile state to the high-profile state after placement of the distal end of the tubular member in the body cavity. The inflation passage operates to maintain pressure in the retention member for prolonged periods of time of up to several weeks.

In another aspect of the invention, the drainage catheter further comprises a foam bolster around the proximal end of the tubular member which may be slightly compressed upon placement of the tubular member to provide a spring force against the retention member in the access tract and to help maintain consistent position of the tubular member. In one aspect of the invention, the tubular member is configured for percutaneous nephrolithotomy while in another aspect of the invention, the tubular member is configured for suprapubic drainage application. The drainage catheter may further comprise a drainage portion having at least one drainage port providing external access for bladder contents via the drainage lumen. The drainage port includes a Luer-lock connection and a drainage bag attachable to the Luer-lock connection. The tubular member may comprise of a soft, silicone material including a radiopaque material to enhance visualization of the catheter. The inflation passage may be connected to a pump or syringe to individually and independently inflate and deflate the retention member. The drainage catheter may further comprise a connector hub at the proximal end including a port and an access lumen plug that operates like a snap-on plug. In another aspect of the invention, the drainage catheter may be used in a veterinary application.

### DESCRIPTION OF THE DRAWINGS

The accompanying drawings, which are included in and constitute a part of this specification, illustrate the embodiments of the invention and, together with the description, explain the features, advantages and principles of the invention. In the drawings:

FIG. 1 is a view of the back of a patient where a tiny incision is made to provide a tunnel directly into the kidney of the patient in a PCNL procedure;

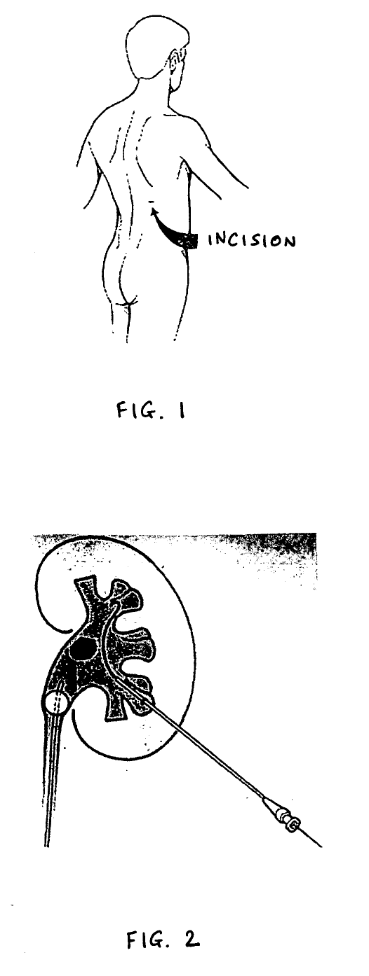

FIG. 2 illustrates a nephroscope used to locate and remove a stone from the kidney;

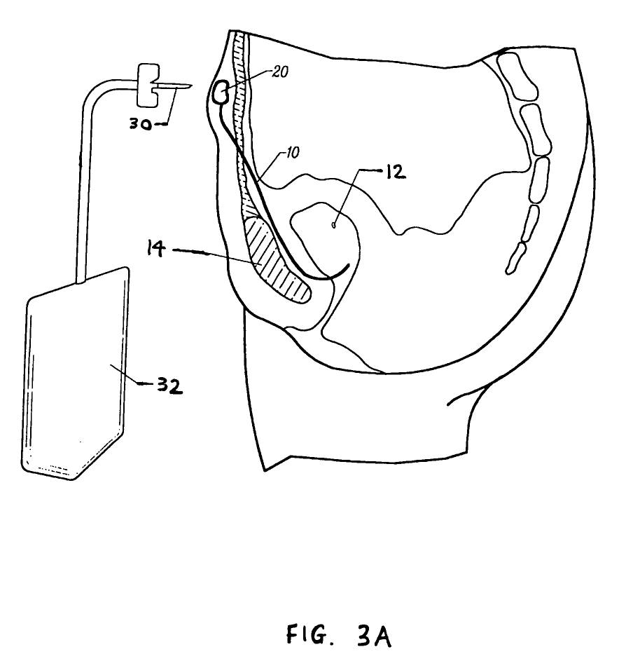

FIGS. 3A and 3B illustrate a suprapubic drainage catheter for percutaneous placement in a bladder of a patient for the removal of urine; and

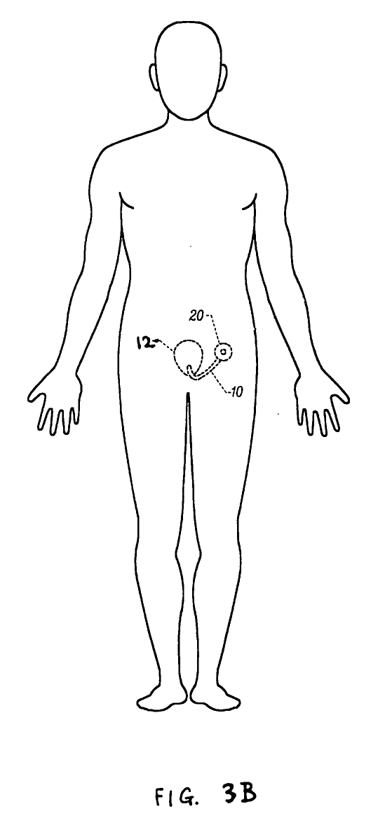

FIGS. 4A and 4B are cross section views of a percutaneous drainage catheter in accordance with an embodiment of the invention.

### DETAILED DESCRIPTION OF THE INVENTION

The following detailed description refers to the accompanying drawings that illustrate the embodiments of the present invention. Other embodiments are possible and modifications may be made to the embodiments without departing from the spirit and scope of the invention. Thus, the following detailed description is not meant to limit the invention.

As discussed above, Percutaneous Nephrolithotomy (PCNL) is a procedure in which the kidney is accessed percutaneously and a 30 Fr cannula is placed into the kidney. The cannula allows the introduction of a nephroscope and other instrumentation with which the surgeon can remove kidney stones. Upon completion of the procedure, the 30 Fr cannula is removed and a drainage catheter is placed in the access tract into the kidney. The drainage catheter may be left in for up to several weeks. A percutaneous drainage catheter may also be placed up to more than 12 weeks in certain situations, for instance when tumors are obstructing the ureter. Placement of the drainage catheter after PNCL provides numerous advantages. For example, the drainage catheter tamponades the access tract to minimize postoperative bleeding. The drainage catheter further provides for drainage of urine, and it provides for ready access to kidney by the surgeon should a complication or emergency arises from the prior procedure.

Drainage catheters are typically catheters in the 6-24 Fr size range and are frequently of the Foley type. As discussed above, Foley catheters have a balloon at the end of the catheter that is inflated to keep the catheter from falling out of the tract. Foley catheters are primarily designed for placement in the bladder through the urethra but have been co-opted for use as a percutaneous drainage catheter. As explained above, these catheters are not very comfortable to the patient and can be very inconvenient. Another area where Foley catheters have been used in a similar fashion is for suprapubic bladder drainage. In this case, the catheter is placed into the bladder through the abdominal wall. The suprapubic catheter is typically placed for patients with spinal cord injuries or with bladder outlet obstructions. Suprapubic catheters primarily serve just to drain urine and must be able to prevent leakage of urine around the shaft of the catheter once placed. As such, there is a need in the art for a drainage catheter that may be percutaneously placed in a patient for an extended period of time (e.g., 12 weeks). The drainage catheter should have an external end that is comfortable to the patient, and the drainage catheter should be easy to clean and maintain. It is contemplated that the drainage catheter may have veterinary applications.

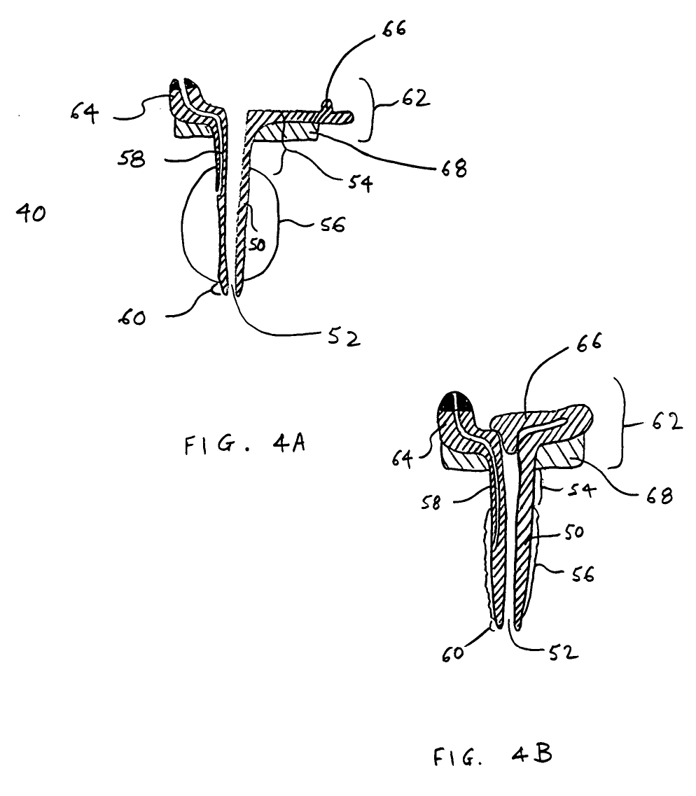

FIGS. 4A and 4B illustrate a drainage catheter 40 in accordance with a preferred embodiment of the invention. The drainage catheter 40 comprises a tubular member 50 having an access or drainage lumen passage 52 extending longitudinally within and a drainage portion 54 having at least one drainage port that provides external access for bladder contents via the drainage lumen passage 52. The drainage port preferably includes a Luer-lock connection that allows attachment to a drainage bag. The drainage catheter 40 further includes a retention member 56 such as an expandable balloon that is connected to the tubular member 50 and positioned distal to the drainage portion 54. The retention member 56 is adapted for movement between a low-profile state facilitating insertion and a high-profile state facilitating retention of the catheter in the body cavity. In particular, the retention member 56 is in a collapsed state around the tubular member 50 of the catheter for percutaneous insertion of the distal portion 60 and the drainage portion 54 of the catheter. Once the distal portion 60 is positioned in the body cavity, the retention member 56 is actuated to an expanded state.

The tubular member 50 includes at least one inflation passage 58 and may include additional passages extending longitudinally therein and communicating with the retention member 56. The tubular member 50 preferably comprises a soft, silicone material including a radiopaque material to enhance visualization of the catheter. The drainage catheter 40 further includes a Docket No. A-2826-AU connector hub 62 including an inflation lumen/port 64 and an access lumen plug 66. The connector hub 62 is designed to protrude minimally from the patient and is capable of sealing the lumen or lumens. The inflation lumen/port 64 may be connected to a commercially available pump or syringe for individually and independently inflating and deflating the retention member 56. The access lumen plug 66 provides easy draining of the body cavity without significant discomfort or risk of infection to the patient. The access lumen plug 66 may be made of the same soft, silicone material including a radiopaque material of the tubular member 50. Moreover, the access lumen plug 66 may be designed to operate like a snap-on plug.

The tubular member 50 and un-inflated retention member 56 have a diameter of approximately 8-10 Fr or 3 mm. The retention member 56 is capable of being inflated to at least slightly more than 30 Fr to tamponade a typical wound created prior to placement. The drainage lumen passage 52 allows the drainage of urine, passage of a guide wire and infusion of liquids, regardless of whether the retention member 56 is inflated or not. In another embodiment of the invention, separate lumens are provided to accommodate each of the above functions. The inflation passage 58, which is separate from the drainage lumen passage 52, allows inflation of the retention member 56 after insertion of the tubular member 50 into the wound tract. The inflation passage 58 maintains pressure in the retention member 56 for prolonged periods of time of up to several weeks. To ease insertion and minimize trauma to surrounding body cavity, a foam bolster 68 may also be provided between the external access points of the tubular member 50 and the patient's skin. The foam bolster 68 provides additional comfort to the patient and may include suture holes to secure the connector hub to the patient's skin. The foam may be slightly compressed when the tubular member 50 is placed in the body cavity, thereby providing a spring force against the retention member 56 in the tract and helping to maintain consistent position of the tubular member 50. The tubular member 50 may be dimensioned for percutaneous nephrolithotomy (e.g., 8-12 cm) or suprapubic (e.g., 4-8 cm) drainage applications.

Many alterations and modifications may be made by those having ordinary skill in the art without departing from the spirit and scope of the invention. For example, it is contemplated that the percutaneous drainage catheter of the invention may be used in veterinary applications. Therefore, it must be understood that the illustrated embodiments have been set forth only for the purposes of examples and that they should not be taken as limiting the invention.

1. A percutaneous drainage catheter, comprising:
a tubular member 50 having a drainage lumen 52 extending from a proximal end and a distal end; and
a retention member 56 formed around the tubular member 50 and is adapted to move between a low-profile state facilitating insertion of the drainage catheter and a high-profile state facilitating retention of the drainage catheter in a body cavity,
wherein the tubular member 50 and the retention member 56 operate to seal and tamponade an access tract in the body cavity.

2. The percutaneous drainage catheter of claim 1, wherein the retention member 56 is disposed at the distal end of the tubular member 50.

3. The percutaneous drainage catheter of claim 1, wherein the retention member 56 is a soft conforming balloon.

4. The percutaneous drainage catheter of claim 1, wherein the tubular member 50 and the retention member 56 in the low-profile state have a diameter of about 8 Fr-10 Fr.

5. The percutaneous drainage catheter of claim 1, wherein the retention member 56 may be expanded to about 30 Fr in the high-profile state.

6. The percutaneous drainage catheter of claim 1, wherein the drainage lumen 52 or additional lumen provide for drainage of urine, passage of a guidewire, and infusion of liquids.

7. The percutaneous drainage catheter of claim 1, wherein the proximal end of the tubular member 50 protrudes minimally from the body cavity.

8. The percutaneous drainage catheter of claim 1, further comprising an inflation passage 58 to actuate the retention member 56 from the low-profile state to the high-profile state after placement of the distal end of the tubular member 50 in the body cavity.

9. The percutaneous drainage catheter of claim 8, wherein the inflation passage 58 maintains pressure in the retention member 56 for prolonged periods of time of up to several weeks.

10. The percutaneous drainage catheter of claim 1, further comprising a foam bolster 68 around the proximal end of the tubular member 50.

11. The percutaneous drainage catheter of claim 10, wherein the foam bolster 68 may be slightly compressed upon placement of the tubular member 50 to provide a spring force against the retention member 56 in the access tract and to help maintain consistent position of the tubular member 50.

12. The percutaneous drainage catheter of claim 1, wherein the tubular member 50 is configured for percutaneous nephrolithotomy.

13. The percutaneous drainage catheter of claim 1, wherein the tubular member 50 is configured for suprapubic drainage application.

14. The percutaneous drainage catheter of claim 1, further comprising a drainage portion 54 having at least one drainage port providing external access for bladder contents via the drainage lumen 52.

15. The percutaneous drainage catheter of claim 14, wherein the drainage port includes a Luer-lock connection.

16. The percutaneous drainage catheter of claim 15, further comprising a drainage bag attachable to the Luer-lock connection.

17. The percutaneous drainage catheter of claim 1, wherein the tubular member 50 comprises a soft, silicone material including a radiopaque material to enhance visualization of the catheter.

18. The percutaneous drainage catheter of claim 8, wherein the inflation passage 58 is connected to a pump or syringe to individually and independently inflate and deflate the retention member 56.

19. The percutaneous drainage catheter of claim 1, further comprising a connector hub 62 at the proximal end including a port 64 and an access lumen plug 66.

20. The percutaneous drainage catheter of claim 19, wherein the access lumen plug 66 provides easy draining of the body cavity.

21. The percutaneous drainage catheter of claim 19, wherein the access lumen plug 66 is formed from a soft, silicone material including a radiopaque material.

22. The percutaneous drainage catheter of claim 19, wherein the access lumen plug 66 operates like a snap-on plug.

23. The percutaneous drainage catheter of claim 1, wherein the drainage catheter is used in a veterinary application.

24. The percutaneous drainage catheter of claim 23, wherein the body cavity is that of an animal.

25. A percutaneous drainage catheter, comprising:
a tubular member 50 having an access lumen 52 extending longitudinally and a drainage portion 54 having at least one drainage port; and
a retention member 56 formed proximally to the tubular member 50 and is adapted to move between a low-profile state facilitating insertion of the drainage catheter and a high-profile state facilitating retention of the drainage catheter in a body cavity,
wherein the tubular member 50 and the retention member 56 operate to seal and tamponade an access tract in the body cavity.

26. The percutaneous drainage catheter of claim 25, wherein the retention member 56 is a soft conforming balloon.

27. The percutaneous drainage catheter of claim 25, wherein the access lumen 52 or additional lumen provide for drainage of urine, passage of a guidewire, and infusion of liquids.

28. The percutaneous drainage catheter of claim 25, wherein the drainage port includes a Luer-lock connection.

29. The percutaneous drainage catheter of claim 28, further comprising a drainage bag attachable to the Luer-lock connection.

30. The percutaneous drainage catheter of claim 25, further comprising a connector hub 62 at a proximal end of the tubular member 50 including a port 64 and an access lumen plug 66.

31. The percutaneous drainage catheter of claim 30, wherein the access lumen plug 66 provides easy draining of the body cavity.

32. The percutaneous drainage catheter of claim 30, wherein the access lumen plug 66 operates like a snap-on plug.

33. The percutaneous drainage catheter of claim 25, wherein the drainage catheter is used in a veterinary application.
